(19) United States
(12) Patent Application Publication
(10) Pub. No.: US 2019/0298507 A1
(43) Pub. Date: Oct. 3, 2019

(54) GRAFT PREPARATION INSTRUMENT

(71) Applicant: SCHWARTZ BIOMEDICAL, LLC, Fort Wayne, IN (US)

(72) Inventors: Herbert E. SCHWARTZ, Fort Wayne, IN (US); Michael James McNICHOLAS, Warrington (GB)

(73) Assignee: SCHWARTZ BIOMEDICAL, LLC, Fort Wayne, IN (US)

(21) Appl. No.: 16/364,832

(22) Filed: Mar. 26, 2019

Related U.S. Application Data
(60) Provisional application No. 62/649,827, filed on Mar. 29, 2018.

(51) Int. Cl. A61F 2/00

(52) U.S. Cl. CPC A61F 2/0077; A61F 2002/0081

(57) ABSTRACT

A graft preparation instrument includes a first mold including first protrusions disposed on a first working surface of the first mold. The first protrusions are separated by first spaces. A second mold includes second protrusions disposed on a second working surface of the second mold. The second protrusions are separated by second spaces. The first and second molds are movable from an open position of the instrument to a closed position of the instrument. In the open position, the first and second working surfaces are separated by an open position distance large enough to place a graft therebetween. In the closed position, the first and second working surfaces are separated by a closed position distance such that the first protrusions are operable to form indentations in a first surface of the graft and the second protrusions are operable to form indentations in a second surface of the graft.

### CROSS-RELATED APPLICATIONS

This Application perfects U.S. Provisional Patent Application No. 62/649,827 filed on Mar. 29, 2018.

### TECHNICAL FIELD

The present invention relates to surgical instruments for handling body tissues that are used as grafts and methods of preparing those grafts during surgery. More specifically, the invention relates to graft preparation instruments that handle grafts used to repair or replace soft tissue during surgery and methods of doing the same.

### BACKGROUND

Grafting refers to a surgical procedure to move tissue (a “graft”) from one site to another in a patient's body, or from a site on a cadaver's body to a site on the patient's body. If a graft is harvested from a patient's body, it is called an autograft. If a graft is harvested from a cadaver, it is called an allograft.

Once a surgeon takes tissue out of a patient's body (i.e., harvests a graft), the graft is subject to a certain amount of preconditioning or preparation in order to be ready to be inserted back into the patient's body. Additionally, the site of the insertion into a patient's body (e.g. a patient's knee) also requires preconditioning or preparation in order to accept the graft.

Accordingly, there is much handling of the graft tissue outside of a patient's body. As such, the risk of contamination of the graft, which can lead to post-operative infection, increases each time the graft is manipulated. To reduce the risk of post-operative infection, grafts are often soaked in a solution of saline and an antibiotic (a “saline solution”) while the insertion site is being prepared. The graft may be soaked in the saline solution in a variety of way. For example, the graft may be disposed in a bowl of the saline solution or may be wrapped in gauzes soaked in the solution.

However, even though the risk of post-operative infection is small, it is always present. Therefore, there is always a need to further reduce the risk of infection from developing in a graft following surgery. Additionally, there is a need for an instrument and method, which will reduce the risk of contamination in a graft while the insertion site is being prepared to accept the graft.

### BRIEF DESCRIPTION

The present invention offers advantages and alternatives over the prior art by providing a graft preparation instrument and method, which temporarily increases the surface area of a graft prior to being placed into a solution of saline and antibiotics. The increased surface area exposes a greater percentage of the graft to the antibiotic in order to reduce the risk of post-operative infection. The surface area of the graft could be temporarily increased by between about 20 to about 50 percent. Further, the instrument and method compresses the bulk volume of the graft prior to being placed into the solution. Once exposed to the solution, the viscoelastic properties of the graft enable the graft to return substantially to its normal shape while soaking in the solution. As such, a portion of the solution is absorbed into the graft to further reduce the risk of post-operative infection.

A graft preparation instrument in accordance with one or more aspects of the present invention includes a first mold including a plurality of first protrusions disposed on a first working surface of the first mold. The first protrusions are separated by a plurality of first spaces. A second mold includes a plurality of second protrusions disposed on a second working surface of the second mold. The second protrusions are separated by a plurality of second spaces. The first and second molds are movable from an open position of the instrument to a closed position of the instrument. In the open position, the first and second working surfaces are separated by an open position distance large enough to place a graft therebetween. In the closed position, the first and second working surfaces are separated by a closed position distance such that the first protrusions are operable to form indentations in a first surface of the graft and the second protrusions are operable to form indentations in a second surface of the graft.

A method of preparing a graft during surgery in accordance with one or more aspects of the present invention includes harvesting a graft from one of a patient's body and a cadaver. A graft preparation instrument is provided. The instrument includes a first mold having a plurality of first protrusions disposed on a first working surface of the first mold. The first protrusions are separated by a plurality of first spaces. The instrument also includes a second mold having a plurality of second protrusions disposed on a second working surface of the second mold. The second protrusions are separated by a plurality of second spaces. The first and second molds are movable from an open position of the instrument to a closed position of the instrument. In the open position, the first and second working surfaces are separated by an open position distance large enough to place a graft therebetween. In the closed position, the first and second working surfaces are separated by a closed position distance such that the first protrusions are operable to form indentations in a first surface of the graft and the second protrusions are operable to form indentations in a second surface of the graft. The method also includes placing the graft between the first and second protrusions when the instrument is in the open position. The instrument is then moved to the closed position. The surface area of the graft is increased by forming indentations in the graft with the first and second protrusions when the instrument is in the closed position.

### DRAWINGS

The invention will be more fully understood from the following detailed description taken in conjunction with the accompanying drawings, in which:

FIG. 1 depicts an embodiment of a perspective view of a graft preparation instrument according to aspects described herein;

FIG. 2 depicts an embodiment of an exploded perspective view of a graft preparation instrument according to aspects described herein;

FIG. 3 depicts an embodiment of a cross-sectional side view of the graft preparation instrument of FIG. 2 taken along the line 3-3 according to aspects described herein;

FIG. 4 depicts an alternative embodiment of a perspective view of a graft preparation instrument according to aspects described herein;

FIG. 5 depicts an embodiment of a perspective front view of a graft preparation instrument in an open position with a graft placed therein;

FIG. 6 depicts an embodiment of a perspective front view of the graft preparation instrument of FIG. 5 in a closed position with the graft placed therein; and

FIG. 7 depicts an embodiment of the graft after having been removed from the graft preparation instrument, the graft temporarily having its surface area increase.

### DETAILED DESCRIPTION

Certain exemplary embodiments will now be described to provide an overall understanding of the principles of the structure, function, manufacture, and use of the methods, systems, and devices disclosed herein. One or more examples of these embodiments are illustrated in the accompanying drawings. Those skilled in the art will understand that the methods, systems, and devices specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The terms “substantially”, “approximately”, “about”, “relatively,” or other such similar terms that may be used throughout this disclosure, including the claims, are used to describe and account for small fluctuations, such as due to variations in processing. For example, they can refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

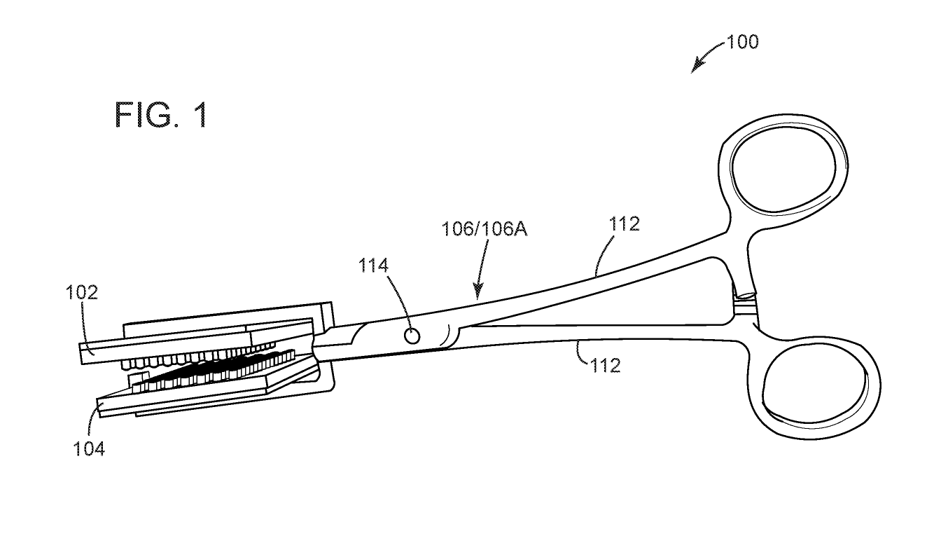

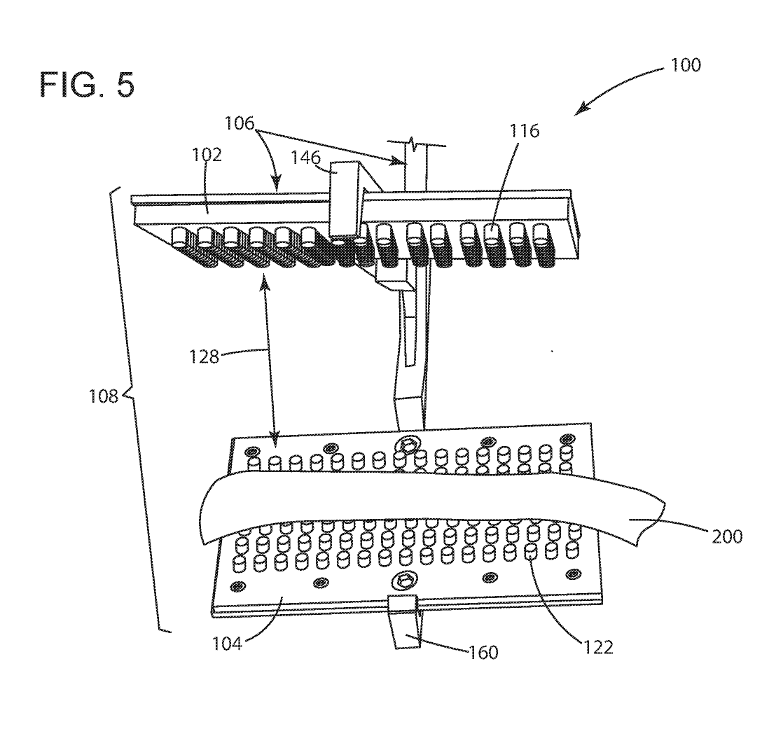

FIGS. 1-4 illustrate various exemplary embodiments of a graft preparation instrument in accordance with aspects described herein. FIGS. 5-7 illustrate various exemplary embodiments of a method of preparing a graft during surgery in accordance with aspects described herein.

Soft tissue grafts, such as autografts from a patient's body or allografts from a cadaver, are generally harvested from ligaments or tendons. The grafts are used to repair or replace other damaged soft tissue in an injured patient, such as the ligaments and tendons that connect the various joints of a human body.

By way of example, the anterior cruciate ligament (ACL), the posterior cruciate ligament (PCL) and other ligaments and soft tissue in the knee joint help to stabilize the joint during articulation activities of normal daily living. These structures hold the upper bone (femur) in relative proximity to the lower bone (tibia) as one flexes and extends their knee during mobility activities or during simple sitting and standing motions. Unfortunately, these ligaments and other soft tissues often become injured (e.g., ruptured, strained, sprained or stretched), causing pain, swelling, and joint instability. Because of the way the knee is loaded, the ACL is often the ligament in the knee that ruptures or gets damaged and needs replacement.

Thus, ligament reconstruction, such as ACL reconstruction, is routinely performed in injured patients. Often the patient's own tissues (autografts) are used to reconstruct the ACL. Typically, an autograft from the hamstrings tendons (such as the gracilis and/or semitendinosis tendons) are used to replace a ruptured or injured ACL.

In order to use a graft tissue, a surgeon must harvest the graft in-situ and prepare the graft for implantation. The preparation procedure often includes significant manipulation of the graft tissue (e.g., trimming, stretching, suturing or the like) outside the body and, thus, subjects the tissue to risk of disease and contamination leading to infection. Currently, after manipulation of a graft, surgeons will often place the prepared graft in a solution of saline and an antibiotic (for example, vancomycin or the like) in an effort to reduce the risk of contamination.

Referring to FIG. 1, an embodiment of a perspective view of a graft preparation instrument 100 is presented. The graft preparation instrument 100 is capable of temporarily increasing the surface area of a graft 200 (best seen in FIGS. 5-7), without damaging the tissue of the graft, prior to being placed into a solution of saline and an antibiotic (not shown). The increased surface area exposes a greater percentage of the graft 200 to the antibiotic in order to further reduce the risk of contamination and post-operative infection. The surface area of the graft 200 could be temporarily increased by between about 20 to about 50 percent. Further, the instrument 100 compresses the bulk volume of the graft 200 prior to being placed into the solution. Once in the solution, the viscoelastic properties of the graft 200 enable the graft to return substantially to its normal shape while soaking in the solution. As such, a portion of the solution is absorbed into the graft 200 to further reduce the risk of contamination.

In one aspect, the graft preparation instrument 100 includes a first mold 102 and a second mold 104 that are operatively connected to a pivotable clamping device 106. The first and second molds 102, 104 may be composed of a biocompatible material that is not toxic, injurious or physiologically reactive to living tissue. Examples of such biocompatible materials would be stainless steel, ultrahigh molecular weight polyethylene polymer or other well-known inert bio-materials.

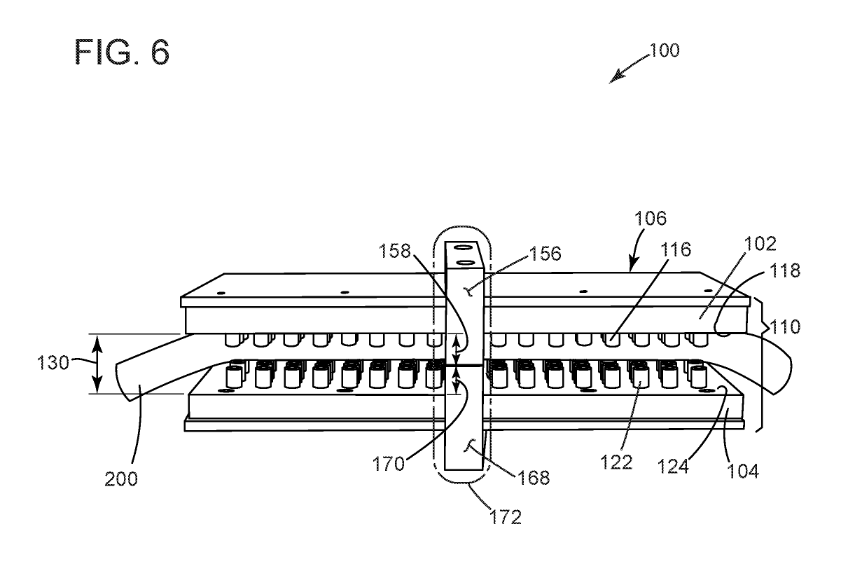

The clamping device 106 is operable to pivot the first and second molds 102, 104 from an open position 108 (best seen in FIG. 5) to a closed position 110 (best seen in FIG. 6). In the specific embodiment of FIG. 1, the clamping device 106 includes a surgical clamp 106A. The surgical clamp 106A includes an elongated pair of scissor handles 112 connected to a pivoting joint 114. The pivoting joint 114 is operatively connected to the first and second molds 102, 104. The clamping device 106 should be stiff enough to transmit the appropriate force from the graft preparation instrument 100 to the graft 200 without damaging the graft preparation instrument 100.

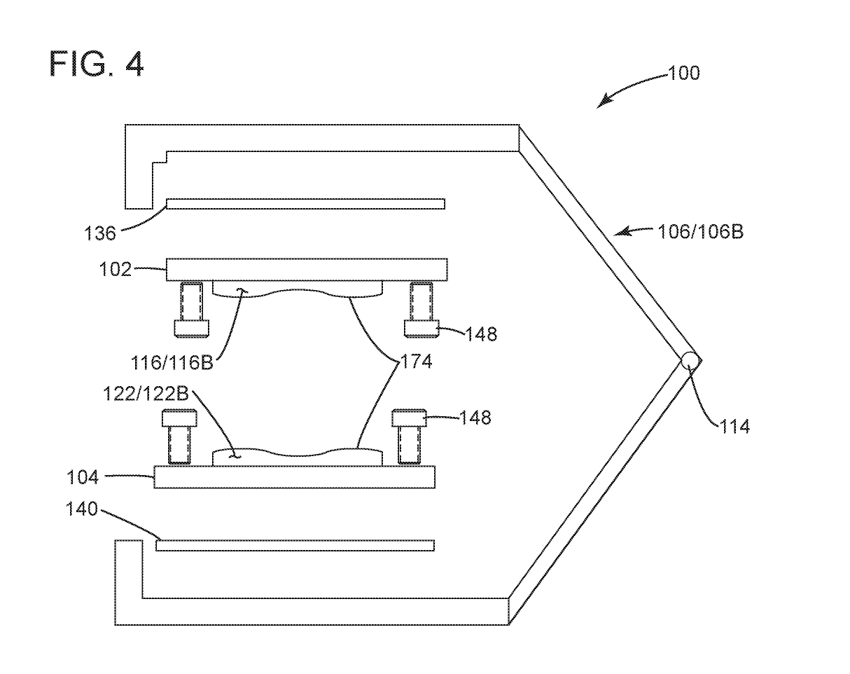

However, though the clamping device 106 of FIG. 1 is configured as a surgical clamp 106A, the clamping device 106 may include various other configurations as well. For example, as illustrated in FIG. 4, the clamping device 106 may have a clam shell configuration 106B.

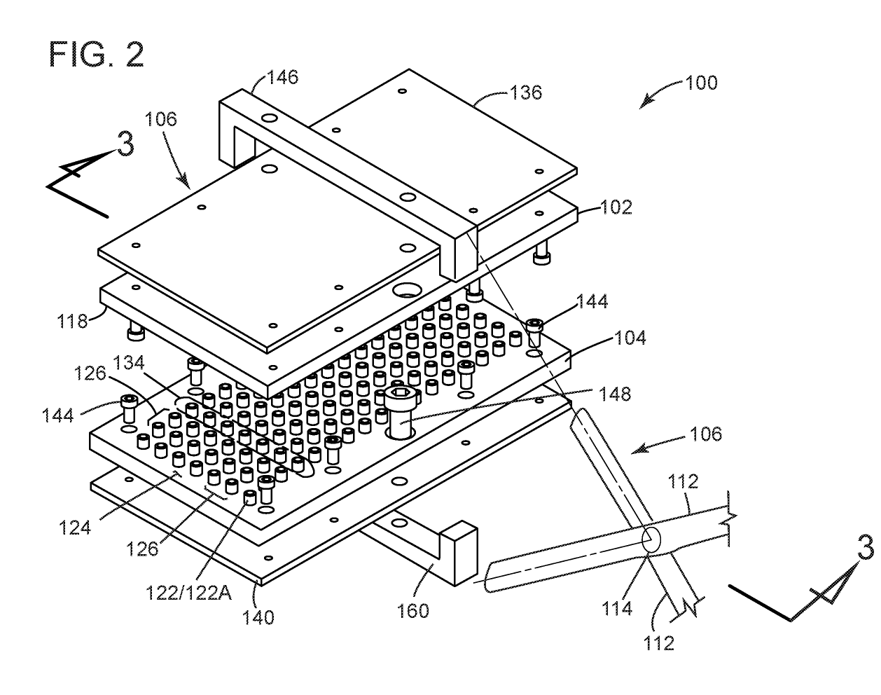

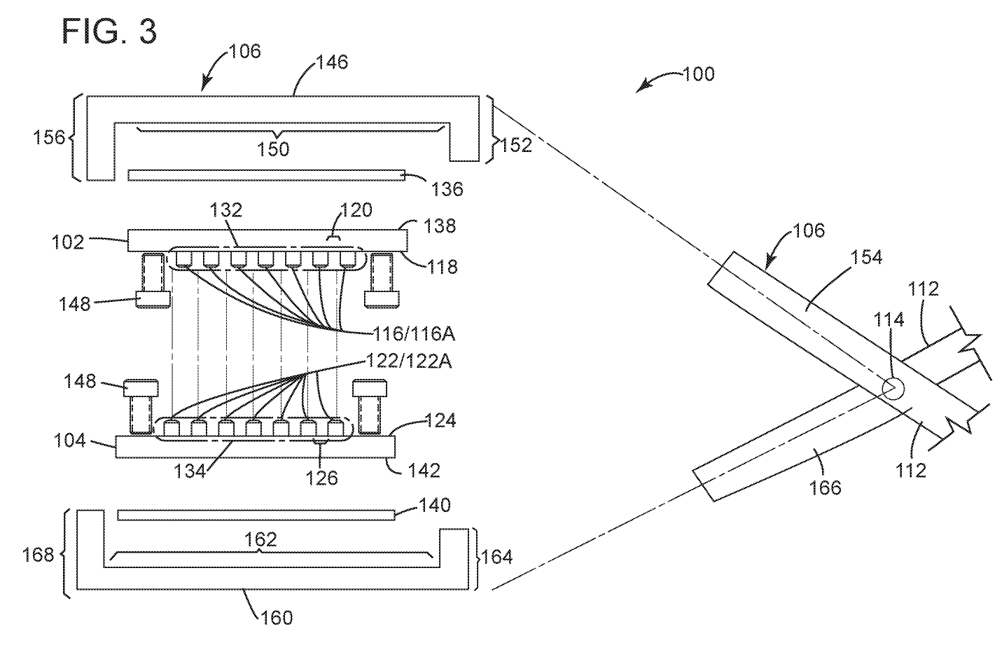

Referring to FIGS. 2 and 3, exploded views of at least a portion of a graft preparation instrument 100 are presented. More specifically, FIG. 2 depicts an embodiment of an exploded perspective view of a graft preparation instrument 100 according to aspects described herein. Additionally, FIG. 3 depicts a cross-sectional side view of the graft preparation instrument of FIG. 2 taken along the line 3-3 according to aspects described herein.

The graft preparation instrument 100 includes the first mold 102 and the second mold 104. The first mold 102 includes a plurality of first protrusions 116 disposed on a first working surface 118 of the first mold 102. The first protrusions 116 are separated by a plurality of first spaces 120. The second mold 104 includes a plurality of second protrusions 122 disposed on a second working surface 124 of the second mold 104. The second protrusions 122 are separated by a plurality of second spaces 126.

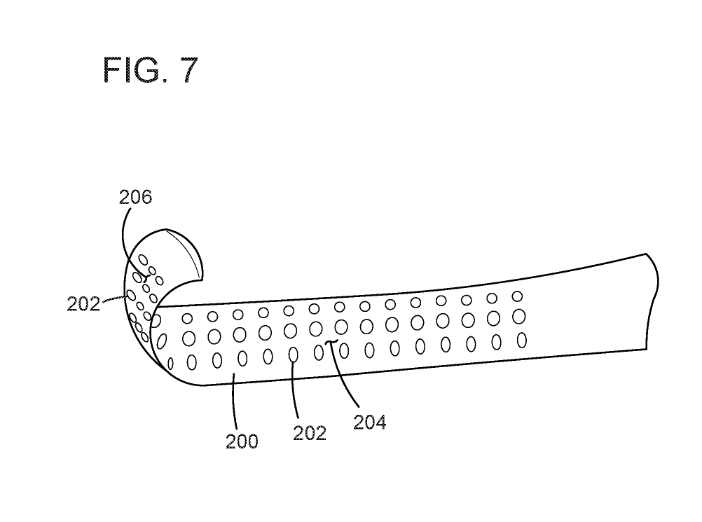

The first and second molds 102, 104 are movable from the open position 108 (best seen in FIG. 5) of the instrument 100 to the closed position 110 (best seen in FIG. 6) of the instrument 100. In the open position 108, the first and second working surfaces 118, 124 are separated by an open position distance 128 (best seen in FIG. 5) large enough to place a graft 200 therebetween. In the closed position 110, the first and second working surfaces 118, 124 are separated by a closed position distance 130 (best seen in FIG. 6). The closed position distance 130 is such that the first protrusions 116 are operable to form indentations 202 in a first surface 204 (best seen in FIG. 7) of the graft 200 and the second protrusions 122 are operable to form indentations 202 in a second surface 206 (best seen in FIG. 7) of the graft 200.

The first and second protrusions 116, 122 may be disposed on their respective molds 102, 104 so that they are staggered apart. Accordingly, when the instrument 100 is in its closed position 110, the first and second protrusions 116, 122 may be meshed together such that the first protrusions 116 fit at least partially between the second spaces 126 and the second protrusions 122 fit at least partially between the first spaces 120.

As illustrated in FIGS. 2 and 3, the first and second protrusions 116, 122 may be in the form of a plurality of cylindrically shaped first and second pegs 116A, 122A respectively. The pegs may be approximately one to two millimeters in diameter and about two millimeters tall in some cases.

Also as illustrated in FIGS. 2 and 3, the first protrusions 116 may be disposed on the first working surface 188 in a plurality of, for example, first parallel rows 132, wherein the first parallel rows 132 are spaced apart by the plurality of first spaces 120. The second protrusions 122 may be disposed on the second working surface 124 in a plurality of, for example, second parallel rows 134, wherein the second parallel rows 134 are spaced apart by the plurality of second spaces 126.

The first and second molds 102, 104 are operatively connected to the pivotable clamping device 106, wherein the clamping device 106 is operable to pivot the first and second molds 102, 104 between the open position 108 and closed position 110.

The particular clamping device 106 illustrated in FIGS. 2 and 3 includes a first base plate 136, which rigidly abuts against a first mounting surface 138 of the first mold 102. The clamping device 106 also includes a second base plate 140, which rigidly abuts against a second mounting surface 142 of the second mold 104. As will be explained in greater detail herein, the first and second base plates 136, 140 are operatively connected to the pivoting joint 114 of the clamping device 106. The first mold 102 and second mold 104 are shown to be flat; however, they could also have a curved contour in order to more closely match the contour of the graft 200.

The first and second base plates 136, 140 are, for example, rigidly affixed to their respective first and second molds 102, 104 with a plurality of first threaded bolts 144. However, any suitable fastening device may be used in lieu of the bolts 144. For example, clips, rivets, adhesive or the like.

The example of the clamping device 106 of FIGS. 2 and 3 also includes a top attachment 146 disposed over the first base plate 136 and rigidly attached thereto with a plurality of second threaded bolts 148. The top attachment 146 includes a top clamping arm 150 extending across a width of the first base plate 136. A top pivot end portion 152 is disposed on one end of the top clamping arm 146. The top pivot end portion 152 rigidly connects the top clamping arm 146 to a first side 154 of the pivoting joint 114.

A top stop portion 156 of the top attachment 146 is disposed on an opposing end of the top clamping arm 150. The top stop portion 156 extends downward perpendicularly from the top clamping arm 150 such that it extends a predetermined distance 158 (best seen in FIG. 6) below the first working surface 118 of the first mold 102.

The clamping device 106 also includes a bottom attachment 160 disposed over the second base plate 140 and is rigidly attached thereto with a plurality of the second threaded bolts 148. The bottom attachment 160 includes a bottom clamping arm 162 extending across a width of the second base plate 140. A bottom pivot end portion 164 is disposed on one end of the bottom clamping arm 162. The bottom pivot end portion 164 rigidly connects the bottom clamping arm 162 to a second side 166 of the pivoting joint 114.

A bottom stop portion 168 of the bottom attachment 160 is disposed on an opposing end of the bottom clamping arm 162. The bottom stop portion 168 extends upward perpendicularly from the bottom clamping arm 162 such that it extends a predetermined distance 170 (best seen in FIG. 6) above the second working surface 124 of the second mold 104.

As can best be seen in FIG. 6, when the first and second molds 102, 104 are in the closed position 110 of the instrument 100, the top and bottom stop portions 156, 168 are engaged to form a mechanical stop 172. The mechanical stop 172 is configured to keep the first and second working surfaces 118, 124 separated by the closed position distance 130. The closed position distance 130 is sized to enable the protrusions 116, 122 to indent the graft 200 without damaging the graft 200.

More specifically, the first and second working surfaces 118, 124 are prevented from closing past the closed position distance 130 so that the staggered first and second protrusions 116, 122 cannot pinch or pierce the tissue of the graft 200. The graft may be made up of bundles of collagen fibers. The mechanical stop 172 prevents the instrument 100 from damaging the bundles while the protrusions move the bundles around to increase the surface area of the graft 200. Also, the stiffness of the clamping device 106 can also be used to control the maximum force that is applied to the graft 200 such that, when the instrument 100 is closed, the graft 200 is not overloaded and damaged.

Referring to FIG. 4, an alternative embodiment of a perspective view of a graft preparation instrument 100 is presented. In this embodiment, the clamping device 106 is in the form of a clam shell design 106B. The clam shell design includes a pivoting joint 114 but does not include the elongated scissor handle 112. As such, the clam shell configuration 106B may be more compact than the surgical clamp configuration 106A of the clamping device 106.

Also shown in FIG. 4 is an alternative embodiment of the first and second protrusions 116, 122. More specifically, the protrusions are in the form of a plurality of parallel first and second rails 116B, 122B. The first and second rails 116B, 122B each have, for example, a substantially sinusoidally shaped top surface 174. The top surfaces 174 provide a series of wave shaped indentations 202 across the graft 200. These rails 116B, 122B could also be, for example, oriented at 90 degrees in the same plane such that they would be parallel to the collagen bundles of the graft 200.

The first and second protrusions 116, 122 have been illustrated herein as cylindrical pegs 116A, 122A (FIGS. 2 and 3), which form circular indentations 202 in a graft 200. The first and second protrusions 116, 122 have also been illustrated herein as elongated rails 116B, 122B (FIG. 4) having a sinusoidally shaped top surface 174, which form wave shaped indentations 202 in the graft 200. However, the protrusions 116, 122 may be configured in other shapes as well to form variously shaped indentations 202. For example, the protrusions 116, 122 may be rectangular pegs which form rectangular indentations. Also by way of example, the protrusions may be rails having a flat top surface, which form elongated or continuous indentations.

FIGS. 5-7 illustrate various exemplary embodiments of a method of preparing a graft during surgery utilizing a graft preparation instrument 100 in accordance with aspects described herein.

Referring to FIG. 5, a perspective view of the graft preparation device 100 in the open position 108 is depicted. More specifically, FIG. 5 depicts an embodiment of a perspective front view of the graft preparation instrument 100, as illustrated in FIGS. 1-3, in an open position 108 with a graft 200 placed therein.

A method of preparing a graft 200 during surgery utilizing the graft preparation device 100 includes harvesting a graft from either a patient's body (for an autograft) or from a cadaver (for an allograft). The graft 200 may be harvested from a ligament or a tendon. More specifically, the tendon may be one of the hamstring tendons, such as the gracillis tendon or the semitendinosis tendon. Alternatively the graft may be harvested from a patella tendon to form a bone-patella tendon-bone graft.

A harvested graft 200, such as from a hamstring tendon, is often long relative to an ACL that it will replace. Accordingly, the harvested graft 200 may be folded, for example, in on itself twice and sutured together to form a quadruple bundle prior to placing the graft 200 into the instrument 100.

Once the quadruple bundle graft 200 is formed, the graft preparation instrument 100 is provided. The graft preparation instrument 100 is opened to its open position 108 and the graft 200 is placed between the first and second protrusions 116, 122.

Referring to FIG. 6, a perspective view of the graft preparation device 100 in the closed position 110 is depicted. More specifically, FIG. 6 depicts an embodiment of a perspective front view of the graft preparation instrument 100 of FIG. 5 in the closed position 110 with the graft 200 place therein.

As illustrated in FIG. 6, once the graft 200 is placed between the first and second protrusions 116, 122, the instrument 100 is moved to the closed position 110. Once in the closed position, the surface area of the graft 200 is increased by forming indentations 202 (best seen in FIG. 7) in the graft 200 with the first and second protrusions 116, 122.

Once the indentations 202 are imprinted into the graft 200, the instrument is moved back to the open position 108 and the graft 200 is removed from the instrument. The graft is then soaked in a solution (not shown) of saline and an antibiotic. For example, the solution may be saline and vancomycin.

Soaking the graft in a solution of saline and antibiotic may be accomplished in a variety of ways. For example, the graft may be placed in a bowl of such solution. Alternatively, the graft may be wrapped in gauzes soaked in the solution.

Additionally, an embodiment of the graft preparation instrument 100 may be capable of being clamped in the closed position 110 such that the entire instrument 100, with the graft 200 disposed therein, may be placed in a solution of saline and antibiotic. Alternatively, the instrument 100 may be constructed to contain a solution of saline and antibiotic within the instrument 100 itself, such that, when the instrument 100 is in the closed position, the graft is simultaneously indented and soaked in the solution.

Referring to FIG. 7, an embodiment of the graft 200 after having been removed from the graft preparation instrument 100 is presented. Advantageously, the graft 100 temporarily has its surface area increased by a significant amount. For example, the surface area of the graft may be increased by from about 20 percent to about 50 percent relative to its surface area before being inserted into the instrument 100. Additionally, the bulk volume of the graft 200 is temporarily compressed with the first and second molds 102, 104 when the instrument 100 is in the closed position 110

To increase the surface area of the graft 200, the first protrusions 116 imprint indentations 202 into a first surface 204 of the graft. Additionally, the second protrusions 122 imprint the indentations 202 into a second surface 206 of the graft 200.

Once the graft 200 is placed in the saline/antibiotic solution, the increased surface area enables more surface contact with the solution and the antibiotic to further reduce the risk of contamination and post-operative infection. Also, the viscoelastic properties of the graft 200 allow the graft to return to substantially its normal shape while soaking in the solution. As the compressed bulk volume increases back to its normal state, a significant portion of the solution is absorbed into the graft 200, much like a compressed sponge absorbs water as it expands. The additional solution absorbed by the graft 200 also helps to further reduce the risk of post-operative infection.

Although the invention has been described by reference to specific embodiments, it should be understood that numerous changes may be made within the spirit and scope of the inventive concepts described. Accordingly, it is intended that the invention not be limited to the described embodiments, but that it have the full scope defined by the language of the following claims.

What is claimed is:

1. A graft preparation instrument, the instrument comprising;
a first mold including a plurality of first protrusions disposed on a first working surface of the first mold, the first protrusions being separated by a plurality of first spaces;
a second mold including a plurality of second protrusions disposed on a second working surface of the second mold, the second protrusions being separated by a plurality of second spaces; and
the first and second molds being movable from an open position of the instrument to a closed position of the instrument, wherein
in the open position, the first and second working surfaces are separated by an open position distance large enough to place a graft therebetween, and
in the closed position, the first and second working surfaces are separated by a closed position distance such that the first protrusions are operable to form indentations in a first surface of the graft and the second protrusions are operable to form indentations in a second surface of the graft.

2. The graft preparation instrument of claim 1, wherein in the closed position, the first and second protrusions are positioned together such that the first protrusions fit at least partially between the second spaces and the second protrusions fit at least partially between the first spaces.

3. The graft preparation instrument of claim 1, wherein the first and second protrusions comprise a plurality of first and second pegs respectively.

4. The graft preparation instrument of claim 1, wherein the first and second protrusions comprise a plurality of first and second rails respectively.

5. The graft preparation instrument of claim 4, wherein the first and second rails include a substantially curved top surface.

6. The graft preparation instrument of claim 1, comprising:
the first protrusions being disposed on the first working surface in a plurality of first parallel rows, the first parallel rows being spaced apart by the plurality of first spaces; and
the second protrusions being disposed on the second working surface in a plurality of second parallel rows, the second parallel rows being spaced apart by the plurality of second spaces.

7. The graft preparation instrument of claim 1, wherein the first and second molds are composed of biocompatible material.

8. The graft preparation instrument of claim 1, comprising:
the first and second molds operatively connected to a pivotable clamping device;
wherein the clamping device is operable to pivot the first and second molds between the open position and closed position.

9. The graft preparation instrument of claim 8, wherein the clamping device includes a mechanical stop, such that when the first and second molds are in the closed position, the mechanical stop is engaged to keep the first and second working surfaces separated by the closed position distance, the closed position distance sized to enable the protrusions to indent the graft without damaging the graft.

10. The graft preparation instrument of claim 9, wherein the clamping device includes:
a first base plate, which rigidly abuts against a first mounting surface of the first mold;
a second base plate, which rigidly abuts against a second mounting surface of the second mold;
the first and second base plates operatively connected to a pivoting joint of the clamping device.

11. The graft preparation instrument of claim 11, comprising:
a top attachment disposed over the first base plate, the top attachment including:
a top clamping arm extending across a width of the first base plate,
a top pivot end portion disposed on one end of the top clamping arm, the top pivot end portion rigidly connecting the top clamping arm to a first side of the pivoting joint, and
a top stop portion disposed on an opposing end of the top clamping arm, the top stop portion extending downward perpendicularly from the top clamping arm such that it extends a predetermined distance below the first working surface of the first mold; and
a bottom attachment disposed over the second base plate, the bottom attachment including:
a bottom clamping arm extending across a width of the second base plate,
a bottom pivot end portion disposed on one end of the bottom clamping arm, the bottom pivot end portion rigidly connecting the bottom clamping arm to a second side of the pivoting joint, and
a bottom stop portion disposed on an opposing end of the bottom clamping arm, the bottom stop portion extending upward perpendicularly from the bottom clamping arm such that it extends a predetermined distance above the second working surface of the second mold;
wherein, when the first and second molds are in the closed position, the top and bottom stop portions are engaged to form the mechanical stop.

12. The graft preparation instrument of claim 8, wherein the clamping device is one of a surgical clamp and a clam shell.

13. A method of preparing a graft during surgery, the method comprising:
harvesting a graft from one of a patient's body and a cadaver;
providing a graft preparation instrument, the instrument comprising:
a first mold including a plurality of first protrusions disposed on a first working surface of the first mold, the first protrusions being separated by a plurality of first spaces,
a second mold including a plurality of second protrusions disposed on a second working surface of the second mold, the second protrusions being separated by a plurality of second spaces, and
the first and second molds being movable from an open position of the instrument to a closed position of the instrument, wherein
in the open position, the first and second working surfaces are separated by an open position distance large enough to place a graft therebetween, and
in the closed position, the first and second working surfaces are separated by a closed position distance such that the first protrusions are operable to form indentations in a first surface of the graft and the second protrusions are operable to form indentations in a second surface of the graft;
placing the graft between the first and second protrusions when the instrument is in the open position;
moving the instrument to the closed position; and
increasing the surface area of the graft by forming indentations in the graft with the first and second protrusions when the instrument is in the closed position.

14. The method of claim 13, comprising:
moving the instrument to the open position;
removing the indented graft from the instrument; and
soaking the graft into a solution of saline and an antibiotic.

15. The method of claim 13, comprising:
folding the harvested graft on itself twice to form a quadruple bundle prior to placing the graft into the instrument.

16. The method of claim 13, wherein the harvesting includes:
harvesting the graft from one of a ligament and a tendon.

17. The method of claim 16, wherein the tendon is at least one of a gracillis tendon, a semitendinosis tendon, and a patella tendon.

18. The method of claim 13, wherein the increasing includes:
increasing the surface area of the graft by from about 20 percent to about 50 percent.

19. The method of claim 13 comprising:
compressing the bulk volume of the graft with the first and second molds when the instrument is in the closed position.

20. The method of claim 14 comprising:
allowing, viscoelastic properties of the graft to return the graft substantially to a normal shape while soaking in the solution; and
absorbing a portion of the solution as the graft returns to its normal shape.
